(19) United States
(12) Patent Application Publication
(10) Pub. No.: US 2022/0241203 A1
(43) Pub. Date: Aug. 4, 2022

(54) METHOD FOR PRODUCTION OF LIPOSOMES

(71) Applicant: UNIVERSIDADE DO MINHO, Braga (PT)

(72) Inventors: DIANA ISABEL PEREIRA GUIMARÃES, AMARANTE (PT); EUGÉNIA SOFIA DA COSTA NOGUEIRA, POUSA (PT); ARTUR MANUEL CAVACO-PAULO, BRAGA (PT)

(21) Appl. No.: 17/607,714

(22) PCT Filed: May 7, 2020
(86) PCT No.: PCT/IB2020/054346

§ 371 Date: Oct. 29, 2021

(30) Foreign Application Priority Data
May 7, 2019 (PT) 115500

(51) Int. Cl. A61K 9/127; A61K 45/06; A61K 47/24; A61K 47/28

(52) U.S. Cl. CPC A61K 9/1271; A61K 45/06; A61K 9/1277; A61K 47/28; A61K 47/24

(57) ABSTRACT

The present disclosure relates to a method for production of liposomes, in order to obtain high encapsulation efficiency of encapsulated agents with a reduced number of production steps, namely avoiding the extrusion step of the classical liposomal production process. The liposomes of the invention are intended to carry a therapeutic agent like an anticancer agent, antioxidant, anti-inflammatory, antipyretic, antibiotic, antiviral, antirheumatic, analgesic, growth-factor, or mixtures thereof.

### TECHNICAL FIELD

The present disclosure relates to a method for production of liposomes, in order to obtain high encapsulation efficiency of encapsulated agents with a reduced number of production steps.

### BACKGROUND ART

Liposomes are defined as artificial microscopic vesicles consisting of an aqueous core surrounded by one or more concentric phospholipid layers (lamellas) [1]. Liposomes have gained extensive attention as carriers for a wide range of therapeutic agents because of being both nontoxic and biodegradable, as they are composed of naturally occurring substances [2]. Liposomes show extensive potential applications as they are able to incorporate hydrophilic (in the aqueous compartment), hydrophobic (within lipidic membrane) and amphiphilic substances (lipid aqueous interface) [3]. Moreover, biologically active materials encapsulated into liposomes are protected from immediate dilution or degradation. For all these reasons liposomes are the most popular nanocarrier systems used since their discovery.

The widespread use of liposomes for several purposes has created the need to develop efficient and reproducible preparation methods with the greatest simplicity as possible. There are different methods for preparation of liposomes, with numerous variants. Because of its simplicity, most laboratory use the lipid thin-film hydration method, first described in 1965 [4]. However, the film method tend to be unsuitable for large scale production. Additionally, there are concerns about the use of chlorinated solvents.

The ethanol injection method is an interesting technique for GMP scaling-up liposomes production. It offers several advantages, e.g. simplicity, GMP friendly solvent, fast implementation and reproducibility, as well as the fact that it does not cause lipid degradation or oxidative alterations. The ethanol injection method was first reported in 1973 by Batzri and Korn [5] as one of the first alternatives for the preparation of small unilamellar vesicles (SUVs) without sonication. By the immediate dilution of the ethanol in the aqueous phase, the lipid molecules precipitate and form bilayer planar fragments. Through energy dissipation in the system (by stirring and/or ultrasonication), the fragments of these lipid bilayers tend to decrease the exposure of the hydrophobic parts of their molecules to the aqueous environment, resulting in the curvature of these fragments which take a quasi-spherical structure. In the following years, several studies have investigated the preparation parameters of the ethanol injection technique (lipid concentration and composition, injection velocity, temperature of both phases, stirring rate, etc.) on the resulting liposome's characteristics (size distribution, zeta potential, drug encapsulation efficiency, etc.) [6].

In the classic ethanolic injection method, the ethanolic phase is in minor percentage comparatively to aqueous phase, usually 5-10%. After ethanol evaporation, the liposomal dispersion is extruded in order to reduce vesicles size. Briefly, classic ethanolic injection method comprises 3 key steps:

1. Injection of lipids (ethanol) in aqueous phase;
2. Extrusion to reduce liposomes size;
3. Remove non-encapsulated agents.

In general, liposomal therapeutic or imaging agents loading is achieved by either passive or active methods:
Passive loading involves dissolution of dried lipid films in aqueous solutions containing the agent of interest. This approach can only be used for water-soluble agents, and the efficiency of loading is often low (smaller than 5%). This method can be used for a wide range of compounds independently of their chemical structure.
Active loading involves the internalization of agents driven by a liposomal transmembrane pH gradient [7]. This process can be extremely efficient (higher than 95%), resulting in high intraliposomal concentrations and minimal wastage of precious chemotherapeutic agents.
   This method (active loading) however requires that molecule have a different protonation state at the extreme pHs of the buffers use inside and outside of liposomes. In such manner a given molecule will diffuse the lipid bilayer when two different pHs are set inside and outside the liposome. Thus, a pH gradient is the driving force to translocate and retain the amphiphilic weak bases and acids [8].

It also reported in the literature the active loading approach for a weakly basic amine therapeutic or imaging agents using a transmembrane ammonium sulfate gradient. In this case, the ammonia gradient drives a pH gradient, leading to active transport of the agent into the liposome. The sulfate then acts as a counterion for the ionized agent, causing it to precipitate within the liposome. This strategy has been applied to the production of liposomal doxorubicin in the case of Doxil. Myocet is another example of liposomal doxorubicin that is remotely loaded, although the pH gradient is established with citric acid [9].

In active loading process, after liposomes preparation with the classic ethanolic injection method, the extra-liposomal phase is removed, and then the agent is added to the extra-liposomal phase and the liposomes are incubated to allow the remote loading process to proceed. Briefly, active loading process comprises 5 key steps:

1. Injection of lipids (ethanol) in aqueous phase;
2. Extrusion to reduce liposomes size;
3. Remove of the extra-liposomal phase;
4. Incubation of liposomes with agents;
5. Remove non-encapsulated agents.

Document WO/2013/084208 (Paulo A. et al., 2013, Liposomes and its production method) describes a method of liposomal production which is the lipidic film hydration method.

Document U.S. Pat. No. 4,752,425A (Martin F. et al., 1988, High-encapsulation liposome processing method) describes a method for production of liposomes that use chloroform. The liposomes produced present 1.5 microns or larger, needing extrusion to size reduction (where the originally encapsulated agent is lost).

Document U.S. Pat. No. 5,549,910A (Szoka F. et al., 1994, Preparation of liposome and lipid complex compositions) describes a method to obtain liposomes containing compounds which exhibit poor solubility in water, alcohols, and halogenated hydrocarbon solvents. In this method the lipids are dissolved in an aprotic solvent solution, which may additionally contain a lower alkanol if needed to solubilize them. This method requires extrusion to obtain liposomes with defined size.

Document US20120171280A1 (Zhang Y, 2011, Method of making liposomes, liposome compositions made by the methods, and methods of using the same) describes a method to obtain liposomes where the aqueous solution comprises ethylenediaminetetraacetic acid (EDTA) to encapsulation ascorbic acid or a salt thereof. The liposome composition have a selected mean particle size diameter of about 200-500 nm.

Document U.S. Pat. No. 5,316,771A (Barenholz, Y. et al., 1994, Method of amphiphatic drug loading in liposomes by ammonium ion gradient) describes active loading of weak amphiphatic drugs into liposomes using transmembrane gradient.

Document U.S. Pat. No. 5,939,096A (Clerc, S. Y. and Barenholz, 1999, Stably encapsulating a weak acid drug in liposomes, at a high concentration) describes liposomes encapsulated with a weak acid drug at a high concentration. The method employed a proton shuttle mechanism involved the salt of a weak acid to generate a higher inside/lower outside pH gradient.

Document WO201364911 relates to methods and compositions for producing lipid-encapsulated negatively-charged therapeutic polymers, such as nucleic acid, proteins and peptides, which are encapsulated within a lipid layer.

Document WO0105374 relates to methods and composition for producing lipid-encapsulated charged therapeutic agent particles, after mixture of preformed lipid vesicles, a charged therapeutic agent (with a charge opposite to the lipid) and a destabilizing agent.

### General Disclosure of the Invention

The method of the description has the advantage of achieving a small molecule encapsulation efficiency in a targeted liposome equal to or better than previous methods without extra processing steps to produce nanoparticles. Polycharged molecules, namely with negative charges in their structure at neutral pHs (5-8) like methotextrate and doxorubicin encapsulate better with this method. Methotextrate is high encapsulation rates and doxorubicin with reduced number of steps. (table 2 and 3)

In the proposed alternative method, a higher encapsulation efficiency of the therapeutic or imaging agent is achieved using a pre-concentration method with an ethanol:aqueous phase at similar volume ratio, and the liposomes may be diluted at the end. The novel proposed method presents a reduced number of steps (only 2) which is desirable in an industrial process. These features are congregated for the first time on the same method, differentiating it from those reported in the literature.

The widespread use of liposomes for several purposes has created the need to develop preparation methods which should be efficient, reproducible and with the greatest simplicity possible. Existing methods remain laborious for industrial scale-up and/or achieving low encapsulation efficiency of the agent of interest. In this way, is imperative the development of a method with a reduced number of steps and that achieve high encapsulation efficiency of the encapsulated agent.

The lipids used to produce the liposomes may be changed or modified to customize the properties of the liposomal surface and membrane layer. There are different classes of lipids, based in their charge: neutral, cationic, and anionic. The addition of organic molecules to the phosphate head group creates a variety of phospholipid species such as phosphatidylethanolamine (PE), phosphatidylserine (PS), phosphatidylglycerol (PG) and phosphatidylcholine (PC). All these lipids could be used in liposomes production.

Unmodified liposomes do not survive long in circulation, as they are removed by macrophages. One of the first attempts to overcome these problems was focused on the manipulation of lipid membrane components in order to modify bilayer fluidity, as example by inclusion of a steroid. In this way, our liposomal formulations may preferentially contain cholesterol (CH), which may vary from a molar ratio of 35-55%, preferably 35-40%. It was demonstrated that incorporation of cholesterol, into liposomes reduces interaction with blood proteins, by causing increased packing of phospholipids in the lipid bilayer.

Furthermore, in a preferential execution was include a synthetic polymer, polyethyleneglycol (PEG) to the liposomes. PEG-containing liposomes showed less binding to blood proteins, reduced RES uptake, and thus prolonged duration of liposomes in the circulatory system. This has extended the blood circulation of conventional liposomes to drug delivery, the conjugated phospholipid DSPE-MPEG was incorporated in lipidic film of these new formulations, in a molar ratio which may vary between 4-12%, preferably 5-10%.

It has also been demonstrated that surface-modified liposomes with gangliosides have a prolonged circulation time in the blood stream compared to non-modified ones. These characteristics are potentially useful for applications of gangliosides in immunotherapies. Several glycolipids have been tested in studies of RES uptake of liposomes after intravenous injection: the glycolipid GM1 (a brain-tissue-derived monosialoganglioside) significantly decreased RES uptake when incorporated on the liposome surface, and the formulation remained in blood circulation for several hours.

Active targeting exploits specific modification of liposomal surface with a targeting ligand, which can lead to their accumulation at the target site or intracellular delivery to target cells. The inclusion of certain ligands in liposomes allows the release of their contents intracellularly by receptor-mediated endocytosis. Targeting agent integration at membrane surface could be achieved by conjugation to phospholipid or fatty acyl chains or incorporated in the lipidic membrane.

There are different methods for preparation of liposomes, with numerous variants. In the ethanol injection method (5% ethanol), a fraction of the aqueous solution with water-soluble substances is passively encapsulated inside the vesicles. The advantage of this method is its simplicity, but only a very small percentage of water-soluble therapeutic or imaging agents can be encapsulated in this way.

In the remote loading, empty liposomes are generally prepared in an initial salt or low pH buffer. The extra-liposomal phase is then removed using dialysis or size exclusion chromatography, or by titrating the pH to slightly basic conditions. Finally, the agent is added to the extra-liposomal phase and the liposomes are incubated to allow the remote loading process to proceed [6]. The number of steps involved makes the production process difficult to scale up, constituting a barrier to further development of this standard approach.

Hence, we focus our efforts in the optimization of agent encapsulation inside liposomes, with a reduced production steps. Indeed, only a low amount of the agent used was encapsulated (e.g. 3-5% to methotrexate drug), being a high amount of agent wasted and this could be an issue in a scale-up process. In order to increase the encapsulated agent numerous conditions were tested in several steps of the production method. Namely: aqueous phase in organic phase containing the phospholipids (instead the opposite), do not remove ethanol, to perform the injection at room temperature instead of at 70° C., to test different speeds of injection and several concentrations of organic phase (5% until 95%).

The present invention consists in a new method of production of liposomes, wherein the hydrophobic components of liposomes are dissolved in ethanol, and injected in an aqueous phase at a rate of approximately 2-4 ml/min. under vigorous agitation. The initial volume ratio ethanol:aqueous phase is 1/1. After evaporation of ethanol or tangential flow filtration the liposomal dispersion should be diluted 1 to 10-fold, to the desirable final concentration.

In an embodiment, pre-concentration method comprises 2 key steps:

Injection of lipids (ethanol) in aqueous phase (1:1 v/v), followed of the dilution;

Remove non-encapsulated agents.

In an embodiment, this method allows the achievement of high encapsulation efficiencies (e.g. ^{˜}40%) for polycharged agent like methotrexate, with a reduced number of steps (only 2). The initial pre-concentration (use of a lower aqueous volume) increase the phospholipid concentration and, consequently allow a higher encapsulation efficiency. Additionally, the use of initial 1:1 of ethanol:aqueous phase volume ratio allows a balance between two phases with different polarities, increasing the encapsulation of the agents.

In an embodiment, the disclosure relates to a method for encapsulating an active ingredient in a liposome comprising the following sequential steps:
preparing an ethanolic phase by mixing hydrophobic molecules of phospholipids and an steroid with ethanol; preferably cholesterol;
preparing an aqueous phase with an active ingredient and a targeting agent in a buffer solution;
obtaining the liposomes by injecting the ethanolic phase in the aqueous phase, at a temperature from around 50° C. to around 80° C., wherein the ethanolic/aqueous phase volume ratio is between 1:1 and 3:2;
removing of the ethanol, by evaporation or tangential flow filtration;
removing the remaining free active ingredient in a suitable way, namely by tangencial flow filtration;
wherein the targeting agent is a peptide that is conjugated with a liposomal component or incorporated in the lipidic membrane.

In another embodiment, the disclosure relates to a method, wherein it further comprises the step of diluting of the liposomal dispersion 1 to 10-fold in further diluted aqueous phase.

In a further embodiment, the disclosure relates to a method, wherein the ethanolic phase is injected at a rate of approximately 2-4 ml/minute.

In a further embodiment, the disclosure relates to a method, wherein the injecting step is performed under agitation.

In a further embodiment, the disclosure relates to a method, wherein the active ingredient is a drug, in particular an anticancer drug, antirheumatic drug, anti-neurodegenerative diseases drug, antioxidant drug, anti-inflammatory, drug antipyretic drug, antibiotic drug, antiviral drug, analgesic drug or combinations thereof.

In another embodiment, the disclosure relates to a method, wherein the targeting agent is a peptide selected from the following list with a degree of identity of at least 90% of the following sequence: SEQ-ID. NO 1, SEQ-ID. NO 2, SEQ-ID. NO 3, or mixtures thereof; comprising at least a sequence 95%, Preferably or at least 96% identical, or at least 97% identical, or at least 98% identical, or at least 99% identical, identical to SEQ-ID. NO 1, SEQ-ID. NO 2, SEQ-ID. NO 3, or mixtures thereof.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. The sequence identity values, which are indicated in the present subject matter as a percentage were determined over the entire amino acid sequence, using BLAST with the default parameters.

In another embodiment, the disclosure relates to a method, wherein the ethanol concentration, relative to the initial aqueous volume, is between 40% and 60%, preferably 50%.

In another embodiment, the disclosure relates to a method, wherein the temperature is 60° C. or 70° C.

In another embodiment, the disclosure relates to a method, wherein the active ingredient is a polycharged molecule containing at least one negative charge at a pH of around 4 to around 7, particularly methotextrate or doxorubicin.

In a further embodiment, the disclosure relates to a method, wherein the aqueous phase is phosphate buffered saline, PBS.

In another embodiment, the disclosure relates to a method wherein the ethanolic phase comprises anionic, neutral or cationic phospholipids.

In a further embodiment, the disclosure relates to a method, wherein the ethanolic phase comprises phosphatidylcholines, phosphatidylethanolamines, phosphatidylserines, phosphatidylglycerols and/or their derivates or mixtures thereof, in particular 1,2-dioleoyl-sn-glycero-3-phosphoethanolamine.

In another embodiment, the disclosure relates to a method wherein the ethanolic phase comprises a steroid, a stealth agent, a targeting agent, or mixture of thereof.

In a further embodiment, the disclosure relates to a method wherein the steroid is cholesterol, and/or their derivate, in particular cholesteryl hemisuccinate.

In another embodiment, the disclosure relates to a method wherein the stealth agent is polyethylene glycol, PEG, or gangliosides

In a further embodiment, the disclosure relates to a method wherein the polyethylene glycol, PEG, is bound to a phospholipid, in particular distearoylphosphatidylethanolamine.

In another embodiment, the disclosure relates to a method wherein the targeting agent is incorporated in the lipidic membrane.

In another embodiment, the disclosure relates to a method wherein the active ingredient is an imaging or therapeutic agent.

Ina further embodiment, the disclosure relates to a method wherein the imaging or therapeutic agent is hydrophobic or hydrophilic.

In another further embodiment, the disclosure relates to a method, wherein the imaging agent is a dye.

### DETAILED DESCRIPTION

The present disclosure relates to a method for production of liposomes, in order to obtain high encapsulation efficiency of encapsulated agents with a reduced number of production steps, namely avoiding the extrusion step of the classical liposomal production process. The liposomes of the invention are intended to carry a therapeutic agent like an anticancer agent, antioxidant, anti-inflammatory, antipyretic, antibiotic, antiviral, antirheumatic, analgesic, growth-factor, or mixtures thereof.

The method of the description has the advantage of achieving a small molecule encapsulation efficiency in a targeted liposome equal to or better than previous methods without extra processing steps to produce nanoparticles.

In order to increase the encapsulated agent, with a reduced production steps, numerous conditions were tested in several steps of the production method. The results demonstrate that the initial percentage of ethanol significantly affected the encapsulation efficiency. A drastic increase in encapsulation efficiency has been noticed as the ethanol volume was higher than the classic 5% (50% relative to the initial aqueous phase), and also using a lower volume of aqueous solution of agent (the sample is afterwards diluted five times to get the usual agent concentration after ethanol evaporation or tangential flow filtration). Vesicles' size has been positively affected by the ethanol volume after this ratio be achieve. Indeed, batches having a higher ethanol volume (>50%) showed larger vesicles (<150 nm) than liposome batches previously produced. These results may be attributed to the slower diffusion of ethanol related to its volume increase in aqueous phase, leading to the formation of higher sized liposomes due to the slow self-assembly of phospholipids. Accordingly, the smaller the vesicles' size, the smaller the aqueous core volume and the lower obtained encapsulation efficiencies, knowing that the hydrophilic agent is mainly encapsulated in the liposome aqueous core [10, 11]. However, a compromise between the encapsulation efficiency and size was obtained using 50% of initial ethanol volume. Liposomes obtained with this percentage of ethanol present small size (<150 nm) and PDI values (<0.1). Moreover, with these conditions, the extrusion process usually needed to decrease, and uniform vesicles' size is perfectly expendable.

### Production of Liposomes

In an embodiment, liposomes composed of DOPE/Cholesterol/DSPE-MPEG (54:36:10, molar ratio) were prepared using the ethanolic injection method. Briefly, lipids (DOPE, cholesterol and DSPE-MPEG) were dissolved in ethanol (5% in the classic ethanol injection method; 50% in the new proposed pre-concentration method, relative to the initial 20% aqueous phase) at 60° C.

The solution was injected under stirring to an aqueous solution (phosphate buffered saline, PBS). This process is done at 70° C., remaining during the necessary time to evaporate all the ethanol volume.

In the classic ethanolic injection method liposomes are extruded to reduce their size. In the pre-concentration method, after ethanol evaporation or tangential flow filtration, liposomal dispersion is diluted five times in PBS (remaining 80% of volume is added). The free therapeutic or imaging agent that was not incorporated into liposomes was removed from the samples after passage through a gel filtration chromatography column (GE Healthcare) with 5 kDa cut-off (PD-10 Desalting Columns containing 8.3 mL of Sephadex G-25 Medium). Hydrophilic therapeutic or imaging agents (e.g. methotrexate and doxorubicin) are present in this aqueous phase in the classic ethanol injection method and in the new proposed pre-concentration method. In remote/active loading, after production in ammonium sulfate (120 mM, pH=8.5), the buffer is changed to Trizma®Base sucrose (10%, w/v, buffered at pH 9.0) and empty liposomes are incubated with the therapeutic or imaging agents.

Characterization of liposomes encapsulating methotrexate: comparative study between several production methods.

| | | | Encapsu- | |
|---|---|---|---|---|
| | | | lation | |
| | Z. average | | effici- | Number of |
| | (d · nm) | PDI | ency (%) | steps |
| Pre-con | | | | 2 (no |
| centration | | | | extrusion) |
| method (50% | 103.1 ± 2.333 | 0.117 ± 0.003 | 43.4 | |
| ethanol | | | | |
| relative to | | | | |
| the initial | | | | |
| aqueous | | | | |
| buffer) | | | | |
| 66% ethanol | 343.3 ± 9.136 | 0.067 ± 0.029 | 41.6 | 2 (no |
| | | | | extrusion) |
| 66% ethanol | 124.7 ± 0.635 | 0.061 ± 0.009 | 8.3 | 3 |
| | | | | (extrusion) |
| 33% ethanol | 99.66 ± 0.095 | 0.240 ± 0.006 | 7.6 | 3 |
| | | | | (extrusion) |
| Classic | 117.7 ± 1.779 | 0.053 ± 0.018 | 5.7 | 3 |
| ethanolic | | | | (extrusion) |
| injection | | | | |
| method (5% | | | | |
| ethanol) | | | | |

TABLE II
Characterization of liposomes encapsulating doxorubicin:
comparative study between several production methods.

| | | | Encapsu- | |
|---|---|---|---|---|
| | | | lation | |
| | Z. average | | effici- | Number of |
| | (d · nm) | PDI | ency (%) | steps |
| Pre-con- | 131.2 ± 1.124 | 0.109 ± 0.010 | 84.9 | 2 (no |
| centration | | | | extrusion) |
| method (50% | | | | |
| ethanol | | | | |
| relative to | | | | |
| the initial | | | | |
| 20% aqueous | | | | |
| buffer) | | | | |
| Classic | 115.1 ± 0.551 | 0.048 ± 0.027 | 85.1 | 3 |
| ethanolic | | | | (extrusion) |
| injection | | | | |
| method (5% | | | | |
| ethanol) | | | | |
| Remote/ | 124.8 ± 1.825 | 0.132 ± 0.020 | 76.1 | 5 |
| active | | | | (extrusion) |
| loading | | | | |

TABLE III
A to Z list of cancer drugs including combination treatments

A
Abemaciclib
Abiraterone Acetate
Abraxane (Paclitaxel Albumin-stabilized Nanoparticle
Formulation)
ABVD
ABVE
ABVE-PC
AC
Acalabrutinib
AC-T
Actemra (Tocilizumab)
Adcetris (Brentuximab Vedotin)
ADE
Ado-Trastuzumab Emtansine
Adriamycin (Doxorubicin Hydrochloride)
Afatinib Dimaleate
Afinitor (Everolimus)
Akynzeo (Netupitant and Palonosetron Hydrochloride)
Aldara (Imiquimod)
Aldesleukin
Alecensa (Alectinib)
Alectinib
Alemtuzumab
Alimta (Pemetrexed Disodium)
Aliqopa (Copanlisib Hydrochloride)
Alkeran for Injection (Melphalan Hydrochloride)
Alkeran Tablets (Melphalan)
Aloxi (Palonosetron Hydrochloride)
Alunbrig (Brigatinib)
Ameluz (Aminolevulinic Acid)
Amifostine
Aminolevulinic Acid
Anastrozole
Apalutamide
Aprepitant
Aranesp (Darbepoetin Alfa)
Aredia (Pamidronate Disodium)
Arimidex (Anastrozole)
Aromasin (Exemestane)
Arranon (Nelarabine)
Arsenic Trioxide
Arzerra (Ofatumumab)
Asparaginase Erwinia chrysanthemi
Atezolizumab
Avastin (Bevacizumab)
Avelumab
Axicabtagene Ciloleucel
Axitinib
Azacitidine
Azedra (Iobenguane I 131)
B
Bavencio (Avelumab)
BEACOPP
Beleodaq (Belinostat)
Belinostat
Bendamustine Hydrochloride
Bendeka (Bendamustine Hydrochloride)
BEP
Besponsa (Inotuzumab Ozogamicin)
Bevacizumab
Bexarotene
Bicalutamide
BiCNU (Carmustine)
Binimetinib
Bleomycin
Blinatumomab
Blincyto (Blinatumomab)
Bortezomib
Bosulif (Bosutinib)
Bosutinib
Braftovi (Encorafenib)
Brentuximab Vedotin
Brigatinib
BuMel
Busulfan
Busulfex (Busulfan)
C
Cabazitaxel
Cabometyx (Cabozantinib-S-Malate)
Cabozantinib-S-Malate
CAF
Calquence (Acalabrutinib)
Campath (Alemtuzumab)
Camptosar (Irinotecan Hydrochloride)
Capecitabine
CAPOX
Carac (Fluorouracil--Topical)
Carboplatin
CARBOPLATIN-TAXOL
Carfilzomib
Carmustine
Carmustine Implant
Casodex (Bicalutamide)
CEM
Cemiplimab-rwlc
Ceritinib
Cerubidine (Daunorubicin Hydrochloride)
Cervarix (Recombinant HPV Bivalent Vaccine)
Cetuximab
CEV
Chlorambucil
CHLORAMBUCIL-PREDNISONE
CHOP
Cisplatin
Cladribine
Clofarabine
Clolar (Clofarabine)
CMF
Cobimetinib
Cometriq (Cabozantinib-S-Malate)
Copanlisib Hydrochloride
COPDAC
Copiktra (Duvelisib)
COPP
COPP-ABV
Cosmegen (Dactinomycin)
Cotellic (Cobimetinib)
Crizotinib
CVP
Cyclophosphamide
Cyramza (Ramucirumab)
Cytarabine
Cytarabine Liposome
Cytosar-U (Cytarabine)
D
Dabrafenib
Dacarbazine
Dacogen (Decitabine)
Dacomitinib
Dactinomycin
Daratumumab
Darbepoetin Alfa
Darzalex (Daratumumab)
Dasatinib
Daunorubicin Hydrochloride
Daunorubicin Hydrochloride and Cytarabine Liposome
Decitabine
Defibrotide Sodium
Defitelio (Defibrotide Sodium)
Degarelix
Denileukin Diftitox
Denosumab
DepoCyt (Cytarabine Liposome)
Dexamethasone
Dexrazoxane Hydrochloride
Dinutuximab
Docetaxel
Doxil (Doxorubicin Hydrochloride Liposome)
Doxorubicin Hydrochloride
Doxorubicin Hydrochloride Liposome
Dox-SL (Doxorubicin Hydrochloride Liposome)
Durvalumab
Duvelisib
E
Efudex (Fluorouracil--Topical)
Eligard (Leuprolide Acetate)
Elitek (Rasburicase)
Ellence (Epirubicin Hydrochloride)
Elotuzumab
Eloxatin (Oxaliplatin)
Eltrombopag Olamine
Emend (Aprepitant)
Empliciti (Elotuzumab)
Enasidenib Mesylate
Encorafenib
Enzalutamide
Epirubicin Hydrochloride
EPOCH
Epoetin Alfa
Epogen (Epoetin Alfa)
Erbitux (Cetuximab)
Eribulin Mesylate
Erivedge (Vismodegib)
Erleada (Apalutamide)
Erlotinib Hydrochloride
Erwinaze (Asparaginase Erwinia chrysanthemi)
Ethyol (Amifostine)
Etopophos (Etoposide Phosphate)
Etoposide
Etoposide Phosphate
Evacet (Doxorubicin Hydrochloride Liposome)
Everolimus
Evista (Raloxifene Hydrochloride)
Evomela (Melphalan Hydrochloride)
Exemestane
F
5-FU (Fluorouracil Injection)
5-FU (Fluorouracil-Topical)
Fareston (Toremifene)
Farydak (Panobinostat)
Faslodex (Fulvestrant)
FEC
Femara (Letrozole)
Filgrastim
Firmagon (Degarelix)
Fludarabine Phosphate
Fluoroplex (Fluorouracil--Topical)
Fluorouracil Injection
Fluorouracil--Topical
Flutamide
FOLFIRI
FOLFIRI-BEVACIZUMAB
FOLFIRI-CETUXIMAB
FOLFIRINOX
FOLFOX
Folotyn (Pralatrexate)
Fostamatinib Disodium
FU-LV
Fulvestrant
Fusilev (Leucovorin Calcium)
G
Gardasil (Recombinant HPV Quadrivalent Vaccine)
Gardasil 9 (Recombinant HPV Nonavalent Vaccine)
Gazyva (Obinutuzumab)
Gefitinib
Gemcitabine Hydrochloride
GEMCITABINE-CISPLATIN
GEMCITABINE-OXALIPLATIN
Gemtuzumab Ozogamicin
Gemzar (Gemcitabine Hydrochloride)
Gilotrif (Afatinib Dimaleate)
Gleevec (Imatinib Mesylate)
Gliadel Wafer (Carmustine Implant)
Glucarpidase
Goserelin Acetate
Granisetron
Granisetron Hydrochloride
Granix (Filgrastim)
H
Halaven (Eribulin Mesylate)
Hemangeol (Propranolol Hydrochloride)
Herceptin (Trastuzumab)
HPV Bivalent Vaccine, Recombinant
HPV Nonavalent Vaccine, Recombinant
HPV Quadrivalent Vaccine, Recombinant
Hycamtin (Topotecan Hydrochloride)
Hydrea (Hydroxyurea)
Hydroxyurea
Hyper-CVAD
I
Ibrance (Palbociclib)
Ibritumomab Tiuxetan
Ibrutinib
ICE
Iclusig (Ponatinib Hydrochloride)
Idarubicin Hydrochloride
Idelalisib
Idhifa (Enasidenib Mesylate)
Ifex (Ifosfamide)
Ifosfamide
IL-2 (Aldesleukin)
Imatinib Mesylate
Imbruvica (Ibrutinib)
Imfinzi (Durvalumab)
Imiquimod
Imlygic (Talimogene Laherparepvec)
Intyta (Axitinib)
Inotuzumab Ozogamicin
Interferon Alfa-2b, Recombinant
Interleukin-2 (Aldesleukin)
Intron A (Recombinant Interferon Alfa-2b)
Iobenguane I 131
Ipilimumab
Iressa (Gefitinib)
Irinotecan Hydrochloride
Irinotecan Hydrochloride Liposome
Istodax (Romidepsin)
Ivosidenib
Ixabepilone
Ixazomib Citrate
Ixempra (Ixabepilone)
J
Jakafi (Ruxolitinib Phosphate)
JEB
Jevtana (Cabazitaxel)
K
Kadcyla (Ado-Trastuzumab Emtansine)
Kepivance (Palifermin)
Keytruda (Pembrolizumab)
Kisqali (Ribociclib)
Kymriah (Tisagenlecleucel)
Kyprolis (Carfilzomib)
L
Lanreotide Acetate
Lapatinib Ditosylate
Lartruvo (Olaratumab)
Lenalidomide
Lenvatinib Mesylate
Lenvima (Lenvatinib Mesylate)
Letrozole
Leucovorin Calcium
Leukeran (Chlorambucil)
Leuprolide Acetate
Levulan Kerastik (Aminolevulinic Acid)
Libtayo (Cemiplimab-rwlc)
LipoDox (Doxorubicin Hydrochloride Liposome)
Lomustine
Lonsurf (Trifluridine and Tipiracil Hydrochloride)
Lupron (Leuprolide Acetate)
Lupron Depot (Leuprolide Acetate)
Lutathera (Lutetium Lu 177-Dotatate)
Lutetium (Lu 177-Dotatate)
Lynparza (liaparib)
M
Marqibo (Vincristine Sulfate Liposome)
Matulane (Procarbazine Hydrochloride)
Mechlorethamine Hydrochloride
Megestrol Acetate
Mekinist (Trametinib)
Mektovi (Binimetinib)
Melphalan
Melphalan Hydrochloride
Mercaptopurine
Mesna
Mesnex (Mesna)
Methotrexate
Methylnaltrexone Bromide
Midostaurin
Mitomycin C
Mitoxantrone Hydrochloride
Mogamulizumab-kpkc
Mozobil (Plerixafor)
Mustargen (Mechlorethamine Hydrochloride)
MVAC
Myleran (Busulfan)
Mylotarg (Gemtuzumab Ozogamicin)
N
Nanoparticle Paclitaxel (Paclitaxel Albumin-stabilized
Nanoparticle Formulation)
Navelbine (Vinorelbine Tartrate)
Necitumumab
Nelarabine
Neratinib Maleate
Nerlynx (Neratinib Maleate)
Netupitant and Palonosetron Hydrochloride
Neulasta (Pegfilgrastim)
Neupogen (Filgrastim)
Nexavar (Sorafenib Tosylate)
Nilandron (Nilutamide)
Nilotinib
Nilutamide
Ninlaro (Ixazomib Citrate)
Niraparib Tosylate Monohydrate
Nivolumab
Nplate (Romiplostim)
O
Obinutuzumab
Odomzo (Sonidegib)
OEPA
Ofatumumab
OFF
Olaparib
Olaratumab
Omacetaxine Mepesuccinate
Oncaspar (Pegaspargase)
Ondansetron Hydrochloride
Onivyde (Irinotecan Hydrochloride Liposome)
Ontak (Denileukin Diftitox)
Opdivo (Nivolumab)
OPPA
Osimertinib
Oxaliplatin
P
Paclitaxel
Paclitaxel Albumin-stabilized Nanoparticle Formulation
PAD
Palbociclib
Palifermin
Palonosetron Hydrochloride
Palonosetron Hydrochloride and Netupitant
Pamidronate Disodium
Panitumumab
Panobinostat
Pazopanib Hydrochloride
PCV
PEB
Pegaspargase
Pegfilgrastim
Peginterferon Alfa-2b
PEG-Intron (Peginterferon Alfa-2b)
Pembrolizumab
Pemetrexed Disodium
Perjeta (Pertuzumab)
Pertuzumab
Plerixafor
Pomalidomide
Pomalyst (Pomalidomide)
Ponatinib Hydrochloride
Portrazza (Necitumumab)
Poteligeo (Mogamulizumab-kpkc)
Pralatrexate
Prednisone
Procarbazine Hydrochloride
Procrit (Epoetin Alfa)
Proleukin (Aldesleukin)
Prolia (Denosumab)
Promacta (Eltrombopag Olamine)
Propranolol Hydrochloride
Provenge (Sipuleucel-T)
Purinethol (Mercaptopurine)
Purixan (Mercaptopurine)
Q
[No Entries]
R
Radium 223 Dichloride
Raloxifene Hydrochloride
Ramucirumab
Rasburicase
R-CHOP
R-CVP
Recombinant Human Papillomavirus (HPV) Bivalent Vaccine
Recombinant Human Papillomavirus (HPV) Nonavalent
Vaccine
Recombinant Human Papillomavirus (HPV) Quadrivalent
Vaccine
Recombinant Interferon Alfa-2b
Regorafenib
Relistor (Methylnaltrexone Bromide)
R-EPOCH
Retacrit (Epoetin Alfa)
Revlimid (Lenalidomide)
Rheumatrex (Methotrexate)
Ribociclib
R-ICE
Rituxan (Rituximab)
Rituxan Hycela (Rituximab and Hyaluronidase Human)
Rituximab
Rituximab and Hyaluronidase Human
Rolapitant Hydrochloride
Romidepsin
Romiplostim
Rubidomycin (Daunorubicin Hydrochloride)
Rubraca (Rucaparib Camsylate)
Rucaparib Camsylate
Ruxolitinib Phosphate
Rydapt (Midostaurin)
S
Sancuso (Granisetron)
Sclerosol Intrapleural Aerosol (Talc)
Siltuximab
Sipuleucel-T
Somatuline Depot (Lanreotide Acetate)
Sonidegib
Sorafenib Tosylate
Sprycel (Dasatinib)
STANFORD V
Sterile Talc Powder (Talc)
Steritalc (Talc)
Stivarga (Regorafenib)
Sunitinib Malate
Sustol (Granisetron)
Sutent (Sunitinib Malate)
Sylatron (Peginterferon Alfa-2b)
Sylvant (Siltuximab)
Synribo (Omacetaxine Mepesuccinate)
T
Tabloid (Thioguanine)
TAC
Tafinlar (Dabrafenib)
Tagrisso (Osimertinib)
Talc
Talimogene Laherparepvec
Tamoxifen Citrate
Tarabine PFS (Cytarabine)
Tarceva (Erlotinib Hydrochloride)
Targretin (Bexarotene)
Tasigna (Nilotinib)
Tavalisse (Fostamatinib Disodium)
Taxol (Paclitaxel)
Taxotere (Docetaxel)
Tecentriq (Atezolizumab)
Temodar (Temozolomide)
Temozolomide
Temsirolimus
Thalidomide
Thalomid (Thalidomide)
Thioguanine
Thiotepa
Tibsovo (Ivosidenib)
Tisagenlecleucel
Tocilizumab
Tolak (Fluorouracil--Topical)
Topotecan Hydrochloride
Toremifene
Torisel (Temsirolimus)
Totect (Dexrazoxane Hydrochloride)
TPF
Trabectedin
Trametinib
Trastuzumab
Treanda (Bendamustine Hydrochloride)
Trexall (Methotrexate)
Trifluridine and Tipiracil Hydrochloride
Trisenox (Arsenic Trioxide)
Tykerb (Lapatinib Ditosylate)
U
Unituxin (Dinutuximab)
Uridine Triacetate
V
VAC
Valrubicin
Valstar (Valrubicin)
Vandetanib
VAMP
Varubi (Rolapitant Hydrochloride)
Vectibix (Panitumumab)
VelP
Velcade (Bortezomib)
Vemurafenib
Venclexta (Venetoclax)
Venetoclax
Verzenio (Abemaciclib)
Vidaza (Azacitidine)
Vinblastine Sulfate
Vincristine Sulfate
Vincristine Sulfate Liposome
Vinorelbine Tartrate
VIP
Vismodegib
Vistogard (Uridine Triacetate)
Vizimpro (Dacomitinib)
Voraxaze (Glucarpidase)
Vorinostat
Votrient (Pazopanib Hydrochloride)
Vyxeos (Daunorubicin Hydrochloride and Cytarabine
Liposome)
W
[No Entries]
X
Xalkori (Crizotinib)
Xeloda (Capecitabine)
XELIRI
XELOX
Xgeva (Denosumab)
Xofigo (Radium 223 Dichloride)
Xtandi (Enzalutamide)
Y
Yervoy (Ipilimumab)
Yescarta (Axicabtagene Ciloleucel)
Yondelis (Trabectedin)
Z
Zaltrap (Ziv-Aflibercept)
Zarxio (Filgrastim)
Zejula (Niraparib Tosylate Monohydrate)
Zelboraf (Vemurafenib)
Zevalin (Ibritumomab Tiuxetan)
Zinecard (Dexrazoxane Hydrochloride)
Ziv-Aflibercept
Zofran (Ondansetron Hydrochloride)
Zoladex (Goserelin Acetate)
Zoledronic Acid
Zolinza (Vorinostat)
Zometa (Zoledronic Acid)
Zydelig (Idelalisib)
Zykadia (Ceritinib)
Zytiga (Abiraterone Acetate)

TABLE IV
A to Z list of drugs used in rheumatoid arthritis therapy

A
Abaloparatide (parathyroid hormone)
Abatacept
Acetaminophen (children and infants)
Acetaminophen 325 mg
Acetaminophen 500 mg
Acetaminophen 650 mg
Acetaminophen with codeine
Acetylsalicylic acid (aspirin)
Actemra
Activella
Actonel
Adalimumab
Addaprin
Advil
Alendronate
Alendronate with vitamin D
Aleve
Allopurinol
Ambien
Ambien CR
Amitriptyline hydrochloride
Amrix
Anacin
Anacin (aspirin free)
Anakinra
Anaprox
Anaprox DS
Apremilast
Arava
Arthrotec
Atelvia
Avinza
Azasan
Azathioprine
Azulfidine
Azulfidine EN-Tabs
B
Baricitinib
Baycadron
Bayer
Belimumab
Benlysta
Betamethasone
Binosto
Boniva
Brisdelle
Bufferin
C
Calcitonin (nasal spray)
Cambia
Canakinumab
Cataflam
Celebrex
Celecoxib
Celestone
CellCept
Cequa (solution)
Certolizumab pegol
Cevimeline
Children's Tylenol
Cimzia
Climara Pro
Clinoril
Cocet
Cocet Plus
Colchicine
Colcrys
Combunox
Conjugated estrogens/bazedoxifene
ConZip
Cortef
Cortisone acetate
Cosentyx
Cyclobenzaprine
Cyclophosphamide
Cyclosporine
Cyclosporine ophthalmic emulslon/solution
Cymbalta
D
Daypro
Denosumab
Dexamethasone
DexPak
Diclofenac
Diclofenac potassium
Diclofenac sodium
Diclofenac Sodium liquid/gel
Diclofenac sodium with misoprostol
Diflunisal
Dolophine
Duavee
Duexis
Duloxetine
Dyspel
E
EC-Naprosyn
Ecotrin
Effexor XR
Embeda
Enbrel
Endocet
Endodan
Estrace
Estratab
Estrogens
Estrogens with progesterone
Etanercept
Etodolac
Evista
Evoxac
Excedrin
F
Febuxostat
Feldene
Fenoprofen calcium
FeverAll
Fexmid
Flexeril
Fluoxetine
Flurbiprofen
Forteo
Fortical
Fosamax
Fosamax Plus D
G
Gabapentin
Gengraf
Genpril
Golimumab
Gralise
H
Horizant
Humira
Hycet
Hydrocet
Hydrocodone bitartrate
Hydrocodone bitartrate with acetaminophen
Hydrocodone bitartrate with ibuprofen
Hydrocortisone
Hydrogesic
Hydroxychloroquine sulfate
Hydroxypropyl cellulose pellets
Hysingla ER
I
Ibandronate
Ibuprofen (over-the-counter)
Ibuprofen (prescription)
Ibuprofen with famotidine
Ilaris
Imuran
Indocin
Indomethacin
Infant's Tylenol
Inflectra (infliximab-dyyb, biosimilar to Remicade)
Infliximab
Intermezzo
I-Prin
Ixekizumab
K
Kadian
Ketoprofen
Kevzara
Kineret
Krystexxa
KS Ibuprofen
L
Lacrisert
Leflunomide
lesinurad
Lorcet
Lortab
Lyrica
M
Magnacet
Maxidone
Meclofenamate sodium
Mediproxen
Medrol
Mefenamic acid
Meloxicam
Menest
Menostar
Methadone hydrochloride
Methadose
Methotrexate
Methylprednisolone
Miacalcin
Millipred
Milnacipran
Mitigare
Mobic
Morphine sulfate
Morphine sulfate oral solution
Morphine sulfate with naltrexone
Motrin
Motrin IB
MS Contin
Mycophenolate mofetil
N
Nabumetone
Nalfon
Naprelan
Naprosyn
Naproxen and esomeprazole magnesium
Naproxen sodium (over-the-counter)
Naproxen sodium (prescription)
Neoral
Neurontin
Norco
O
Olumiant
Opana
Oramorph SR
Orapred
Orencia
Otezla
Otrexup
Oxaprozin
Oxycodone
Oxycodone hydrochloride with acetaminophen
Oxycodone with aspirin
Oxycodone with ibuprofen
OxyContin
Oxymorphone hydrochloride
P
Paroxetine
Paxil
PediaCare Fever Reducer/Pain Reliever
Pediapred
Pegloticase
Pennsaid
Percocet
Percodan
Pexeva
Pilocarpine
Piroxicam
Plaquenil
Ponstel
Prednisolone
Prednisone
Prednisone Intensol
Pregabalin
Prelone
Premphase
Prempro
Primlev
Probenecid
Probenecid and colchicine
Prolia
Prozac
R
Raloxifene hydrochloride
Rasuvo
Rayos
Reclast
Remicade
Renflexis (infliximab-abda, biosimilar to Remicade)
Reprexain
Restasis (emulsion)
Rheumatrex
Risedronate sodium
Rituxan
Rituximab
Roxicet
Roxicodone
Rybix ODT
Ryzoit
S
Salagen
Sandimmune
Sarafem
Sarilumab
Savella
secukinumab
Sertraline
Simponi, Simponi Aria
Stelara
Sulfasalazine
Sulfazine
Sulfazine EC
Sulindac
T
Taltz
Teriparatide (parathyroid hormone)
TH Ibuprofen
Therafeldamine
Tivorbex
Tocilizumab
Tofacitinib (extended release)
Tofacitinib (immediate release)
Tolmetin sodium
Tramadol
Tramadol (extended release)
Tramadol with acetaminophen
Trexall
Tylenol Arthritis Pain
Tylenol Extra Strength
Tylenol Regular Strength
Tylenol with Codeine No. 2
Tylenol with Codeine No. 3
Tylenol with Codeine No. 4
Tymlos
U
Uloric
Ultracet
Ultram
Ultram-ER
Ustekinumab
V
Venlafaxine
Veripred 20
Vicodin
Vicoprofen
Vimovo
Vivlodex
Voltaren
Voltaren XR
X
Xatmep
Xeljanz
Xeljanz XR
Xodol
Xolox
Z
Zamicet
Zipsor
Zohydro ER
Zoledronic acid
Zoloft
Zolpidem
Zolvit
Zometa
Zorvolex
Zurampic
Zydone
Zyloprim

TABLE IV
A to Z list of dyes

A
Acetaldehyde-2,4-dinitrophenylhydrazone analytical standard,
for environmental analysis
S-Acetamido-3-[4-[3-[4-(2,4-di-tert-
pentylphenoxy)butylcarbamoyl]-4-hydroxy-1-
naphthoxy]phenylazo]-4-hydroxy-2,7-naphthalenedisulfonic acid
disodium salt Dye content 90%
Acetone-2,4-dinitrophenylhydrazone environmental standard,
99%
3-Acetylnoradamantane technical grade, 85%
Acid Blue 25 Dye content 45%
Acid Blue 29 Dye content 40%
Acid Blue 80 Dye content 40%
Acid Blue 113 Dye content 50%
Acid Blue 129 Dye content 25%
Acid Fuchsin used in tissue staining
Acid Fuchsin calcium salt certified by the Biological Stain
Commission, Dye content ≥60%
Acid Green 25 Dye content ≥60%
Acid Orange 8 Dye content 65%
Acid Red 1 Dye content 60%
Acid Red 183 Dye content 30%
Acid Violet 7 Dye content 40%
Acid Yellow 17 Dye content 60%
Acid Yellow 25 Dye content 40%
Acridine 57%
Acridine Orange hemi(zinc chloride) salt Dye content 90%
Acridine Orange hydrochloride hydrate ≥98% (HPLC)
Acridine Orange hydrochloride solution 10 mg/mL in H2O
Acridine Orange 10-nonyl bromide
Acriflavine
Acriflavine hydrochloride
Adrenochrome
ADVASEP ™-7 solid
Alcian Blue solution 1% in 3% acetic acid, pH 2.5
Alcian Blue 8GX powder
Alcian Blue 8GX certified by the Biological Stain Commission
Alcian Blue 8GX for microscopy (Bact., Bot., Hist.)
Alcianblue 8GX solution for microscopy, 1% in solution (in 3%
acetic acid)
Alcian Blue, pyridine variant Dye content ≥85%
Alexa Fluor 350
Alexa Fluor 405
Alexa Fluor 488
Alexa Fluor 532
Alexa Fluor 546
Alexa Fluor 555
Alexa Fluor 568
Alexa Fluor 594
Alexa Fluor 647
Alexa Fluor 680
Alexa Fluor 750
Alizarin Dye content 97%
Alizarin Blue Black B
Alizarin Red S certified by the Biological Stain Commission
Alizarin Yellow GG Dye content 50%
Allophycocyanin
Allura Red AC Dye content 80%
Amaranth Dye content 85-95%
p-Amidinophenyl p-(6-amidmo-2-indolyl)phenyl ether
dihydrochloride
1-Aminoanthraquinone 97%
4-Amino-1,1′-azobenzene-3,4′-disulfonic acid monosodium salt
Dye content 95%
3-Amino-3-deoxydigoxigenin hemisuccinamide, succinimidyl
ester
N-(4-Amino-2,5-diethoxyphenyl)benzamide
2-Amino-5,6-dimethylbenzimidazole 97%
4-Amino-3,5-disulfo-1,8-naphthalic anhydride dipotassium salt
N-(5-Aminopentyl)biotinamide trifluoroacetate salt solid
2-(3-Aminophenylsulfonyl)ethanol hydrochloride 97%
4-Ammophthalonitrile 98%
8-Aminopyrene-1,3,6-trisulfonic acid trisodium salt ≥96.0%
HPCE), solid
Aniline Blue diammonium salt certified by the Biological Stain
Commission
Aniline Blue solution 2.5% in 2% acetic acid
N-[(3-(Anilinomethylene)-2-chloro-1-cyclohexen-1-
yl)methylene]aniline monohydrochloride 94%
8-Anilino-1-naphthalenesulfonic acid
8-Anilino-1-naphthalenesulfonic acid hemimagnesium salt
hydrate for fluorescence, ≥95% (perchloric acid titration)
Anisaldehyde solution
Anthrone ACS reagent, 97%
Arsenazo III calcium-sensitive dye
Astrazon Orange G
Auramine O Dye content ≥80%, certified by the Biological Stain
Commission
1-Azidoadamantane 97%
Azobenzene 98%
Azocarmine G
Azomethine-H monosodium salt hydrate ~95%
Azophloxine for microscopy (Hist.)
Azure B certified by the Biological Stain Commission
Azure B prepared by direct synthesis
Azure A chloride Dye content ≥70%
Azure A chloride certified by the Biological Stain Commission
Azure A eosinate
Azure B eosinate
Azure II powder
Azure II eosinate
Azure B tetrafluoroborate Dye content 95%
B
Bacteriochlorophyll from Rhodopseudomonas sphaeroides
Basacryl Red GL Dye content 19%
Basic Blue 3 Dye content 25%
Basic Blue 41 Dye content 40%
Basic Fuchsin for microscopy (Bact., Bot., Hist.), indicator (pH 1.0-
3.1)
Basic Fuchsin special for flagella, certified
Basic Fuchsin certified by the Biological Stain Commission, Dye
content ≥88%
Basic Fuchsin Dye content >85%
Bathocuproinedisulfonic acid disodium salt for
spectrophotometric det. of Cu, Fe
Bathophenanthrolinedisulfonic acid disodium salt hydrate ≥95%
Benzaldehyde-2,4-dinitrophenylhydrazone environmental
standard, 99%
Benzaldehyde tosylhydrazone 98%
Benzidine ≥98.0% (N)
Benzidine ISOPAC ®, ≥98.0% (N)
Benzidine dihydrochloride ≥99% (titration)
Benzophenone imine 95%
N-Benzylideneaniline 99%
N-Benzylidenebenzenesulfonamide 97%
N-Benzylidenebenzylamine contains 100 ppm MEHQ as stabilizer,
99%
Biotin-4-Fluorescein
bisBenzimide H 33258 ≥98% (HPLC and TLC)
bisBenzimide H 33342 trihydrochloride ≥98% (HPLC and TLC)
Bis(cyclohexanone)oxaldihydrazone
Bis(1,3-dibutylbarbituric acid) trimethine oxonol ≥95% (HPLC)
N,N′-Bis(2,5-di-tert-butylphenyl)-3,4,9,10-perylenedicarboximide
Dye content 97%
1,3-Bis[4-(dimethylamino)phenyl]-2,4-
dihydroxycyclobutenediylium dihydroxide, bis(inner salt) Dye
content 90%
[4-[Bis(2-hydroxyethyl)amino]phenyl]-1,1,2-
ethylenetricarbonitrile 98%
Bismarck Brown R for microscopy (Bact., Hist.)
Bismarck Brown Y certified by the Biological Stain Commission,
Dye content 50%
N,N′-Bis(salicylidene)-1,2-phenylenediamine 97%
Brilliant Black BN Dye content 60%
Brilliant Blue G solution Concentrate
Brilliant Blue R Dye content ~50%, Technical grade
Brilliant Blue R pure
Brilliant Blue G pure
Brilliant Blue R Concentrate suitable for SDS-PAGE, methanol
solution
Brilliant Blue R Staining Solution ethanol solution
‘Brilliant Cresyl blue’ for microscopy (Vit.), mixture of toluidine
blue and waterblue
Brilliant Cresyl Blue ALD Certified by the Biological Stain
Commission
Brilliant Cresyl Blue ALD certified by the Biological Stain
Commission
Brilliant Green Dye content ~90%
Brilliant Green certified by the Biological Stain Commission
Brilliant Yellow Dye content ≥50%
Bromaminic acid sodium salt
Bromobimane ≥97%
4′-Bromo-(1,1′-biphenyl)-4-ol 97%
Bromochlorophenol Blue Dye content 95%
Bromocresol Green ACS reagent, Dye content 95%
Bromocresol Green sodium salt crystalline
Bromocresol Green sodium salt ACS reagent, Dye content 90%
Bromocresol Green sodium salt solution 0.04 wt. % in H2O
Bromocresol Green/Methyl Red, mixed indicator solution in
methanol
Bromocresol Green Sultone Form for microscopy (Bot., Hist.,
Vit.), indicator (pH 3.8-5.4)
Bromocresol Purple BioReagent, suitable for indicator, Dye
content 90%
Bromocresol Purple Technical grade
Bromocresol Purple for microscopy (Hist., Vit.), indicator (pH 5.2-
6.8)
Bromocresol Purple sodium salt indicator grade, Dye content
90%
Bromocresol Purple solution 0.04 wt. % in H2O
Bromophenol Blue
Bromophenol Blue ACS reagent
Bromophenol Blue sodium salt for molecular biology, for
electrophoresis
Bromophenol Blue sodium salt
Bromophenol Blue sodium salt Dye content 90%, ACS reagent
Bromophenol Blue solution 0.04 wt. % in H2O
2-(5-Bromo-2-pyridylazo)-5-(diethylamino)phenol 97%
Bromothymol Blue ACS reagent, Dye content 95%
Bromothymol Blue sodium salt powder
Bromothymol Blue sodium salt ACS reagent
Bromothymol Blue sodium salt solution 0.04 wt. % in H2O
2-Bromo-1,1,3-trimethoxypropane 95%
Bromoxylenol Blue indicator grade, Dye content 95%
6-tert-Butyl-2,3-naphthalenedicarbonitrile 94%
4-tert-Butylphthalic anhydride 95%
4-tert-Butylphthalonitrile 98%
N-tert-Butyl-α-(2-sulfophenyl)nitrone sodium salt 95%
Sulfobromophthalein disodium salt hydrate used to study
hepatocyte transport functions
C
Calcein Used for the fluorometric determination of calcium and
EDTA titration of calcium in the presence of magnesium.
Calcein AM solution 4 mM in DMSO, ≥90% (HPLC), solution
Calcium Ionophore A23187 mixed calcium magnesium salt
Approximate 1:1 molar ratio Ca:Mg. Actual content given on
label.
Calconcarboxylic acid
Calmagite indicator grade
Calmagite triethanolammonium salt
Canada balsam Mounting medium for microscopy
Carbazole ≥95% (GC)
Carbol Fuchsin
Carbostyril 124 99%
b-Carboxy-4′,5′-dichloro-2′,7^{′}-dimethoxyfluorescein N-
hydroxysuccinimide ester
5-Carboxyfluorescein 99% (HPLC)
6-Carboxyfluorescein ~97% (HPLC)
5-Carboxyfluorescein diacetate ~95% (HPLC)
5(6)-Carboxyfluorescein diacetate Mixed isomers ≥90% (HPLC)
N-Carboxymethyl-6-(2,2-dicyanovinyl)-1,2,3,4-
tetrahydroquinoline ≥98% (HPLC)
5-Carboxytetramethylrhodamine
Cardiogreen polymethine dye
Carmine powder
Carmine certified by the Biological Stain Commission
Carminic acid
Celestine blue Dye content 80%
Chicago Sky Blue 6B powder
Chlorazol Black
1-Chloro-9,10-bis(phenylethynyl)anthracene 99%
2-Chloro-5-methylaniline 99%
2′-Chloro-S′-methylbenzanilide 97%
S-Chloro-2-methylindole 97%
2-Chloro-6-nitrobenzaldehyde 97%
Chlorophenol Red indicator grade
Chlorophyll a from Anacystis nidulans algae
Chlorophyll b from spinach ≥90% (HPLC), ≤0.5% Chlorophyll a
4-Chloro-7-sulfobenzofurazan ammonium salt
Chromeazurol B
Chromeazurol S Dye content 50%
Chromotrope 2R suitable for modified Gomori Trichrome stain
Chromotrope FB Dye content 50%
Chrysin 97%
Chrysoidine G for microscopy (Bact., Bot., Vit.)
Chrysophenine Dye content 65%
Cibacron Brilliant Yellow 3G-P
Clobetasone butyrate ≥98%
Collodion solution for microscopy, 2% in amyl acetate
Congo Red certified by the Biological Stain Commission, BioXtra
Congo Red Dye content ≥35%
Coomassie Violet R200 Dye content 50%
Coproporphyrin I tetramethyl ester ≥90% (HPLC)
Coumarin 6 98%
Coumarin 7 98%
Coumarin 314 Dye content 97%
Coumarin 334 Dye content 99%
Coumarin 343 Dye content 97%
o-Cresolphthalein indicator grade
o-Cresolphthalein Complexone powder
m-Cresol Purple indicator grade, Dye content 90%
m-Cresol Purple sodium salt Dye content 90%
Cresol red indicator grade, Dye content 95%
Cresol Red sodium salt indicator grade
Cresyl Violet acetate certified by the Biological Stain Commission
Crocein Scarlet 7B
Croconic acid 98%
Crotonaldehyde-2,4-dinitrophenylhydrazone analytical standard,
for environmental analysis
Crystal Ponceau 6R
Crystal Violet Dye content ≥90%
Crystal Violet ACS reagent, ≥90.0% anhydrous basis
Crystal Violet certified by the Biological Stain Commission
Crystal violet solution 1%, aqueous solution
Crystal Violet lactone Dye content 97%
Curcumin from Curcuma longa (Turmeric), powder
N-[3-Cyano-3-[4-(dicyanomethyl)phenyl]-2-propenylidene]-N-
ethyl-ethaniminium inner salt
9-Cyano-N,N,N′-triethylpyronine-N′-caproic acid N-
hydroxysuccinimide ester chloride ≥85% (HPLC)
9-Cyano-N,N,N′-triethylpyronine-N′-caproic acid N4-
(maleimidoethyl)piperazide chloride ≥80% (HPLC)
Cyclohexanone 2,4-dinitrophenylhydrazone ≥99%
D
DAPI, dilactate ≥98% (HPLC)
Darrow Red certified by the Biological Stain Commission, Dye
content ≥65%
o-Dianisidine dihydrochloride ≥95%
o-Dianisidine dihydrochloride Suitable for use in glucose
determination
o-Dianisidine dihydrochloride for enzymic, spectrophotometric
determination, vial of 5 mg
4,5-Dibromobenzene-1,2-diol 90%, technical grade
4′,5′-Dibromofluorescein Dye content 95%
2,5-Dibromo-6-isopropyl-3-methyl-1,4-benzoquinone
3,6-Dibutoxy-1,2-benzenedicarbonitrile 97%
2,5-Dibutoxy-4-(4-morpholinyl)benzenediazonium
tetrafluoroborate Dye content 95%
1,4-Dibutoxy-2,3-naphthalenedicarbonitrile 99%
4-(Dibutylamino)benzaldehyde 98%
4-(2-(6-(Dibutylamino)-2-naphthalenyl)ethenyl)-1-(3-
sulfopropyl)pyridinium hydroxide inner salt ≥95% (HPLC), solid
N,N-Dibutylaniline 97%
2,4-Dichlorobenzenediazonium 1,5-naphthalenedisulfonate
hydrate
3,6-Dichloro-1,2-benzenedithiol 95%
1,2-Dichloro-4,5-dinitrobenzene 98%
2′,7′-Dichlorofluorescein ~90% (TLC), crystalline
2′,7′-Dichlorofluorescein ACS reagent
2,6-Dichloroindophenol sodium salt hydrate suitable for vitamin
C determination, BioReagent
2,6-Dichloroquinone-4-chloroimide
6-(2,2-Dicyanovinyl)-N-(2-hydroxyethyl)-1,2,3,4-
tetrahydroquinoline
trans-4-(Diethylamino)cinnamaldehyde 98%
7-Diethylamino-3-(4-maleimidophenyl)-4-methylcoumarin ≥95%
(HPLC), solid
1,1′-Diethyl-2,2′-carbocyanine iodide 97%
1,1′-Diethyl-2,2′-cyanine iodide 97%
1,1′-Diethyl-4,4′-cyanme iodide
N,N-Diethyl-p-phenylenediamine oxalate salt
Diethyl 3,4-pyridinedicarboxylate 97%
3,3′-Diethylthiacyanine iodide Dye content ~97%
Dihydroethidium ≥95%
Dihydrorhodamine 123 ≥95%
2′,4′-Dihydroxy-3′-methylacetophenone technical grade, 90%
1,3-Dihydroxynaphthalene ≥99%, crystalline
1,4-Dihydroxy-2,3-naphthalenedicarbonitrile 97%
4,7-Dihydroxy-1,10-phenanthroline Dye content ≥30%
1,3-Diiminobenz[f]isoindoline 95%
1,3-Diiminoisoindoline 97%
3,3′-Dimethoxybenzidine dihydrochloride technical grade
(2,5-Dimethoxyphenyl)acetyl chloride 99%
2,4-Dimethoxytoluene 99%
4-(Dimethylamino)benzaldehyde suitable for histochemical
demonstration of nitro blue tetrazolium reduction in neutrophils
4-(Dimethylamino)cinnamaldehyde chromogenic reagent for
indoles and flavanols
4-Dimethylamino-2-methylazobenzene
2-[4-(Dimethylamino)styryl]-1-ethylpyridinium iodide ≥99%
(HPLC), solid
2-[4-(Dimethylamino)styryl]-N-methylbenzoxazolium perchlorate
N,N-Dimethyl-4,4′-azodianiline 97%
N,N′-Dimethyl-9,9′-biacridmium dinitrate used as
chemiluminescent reagent
N,N-Dimethylindoaniline Dye content 97%
3,3-Dimethyl-2-methyiene-1-phenylindoline
N,N-Dimethyl-1-naphthylamine ≥98.0% (GC)
N,N-Dimethyl-4-nitrosoaniline 97%
N,N-Dimethyl-p-phenylenediamine dihydrochloride suitable for
peroxidase test, ≥99.0% (titration)
1,4-Dimethylpyridinium iodide 99%
1,4-Dimethylpyridinium p-toluenesulfonate 98%
3,5-Dinitroaniline 97%
4,4′-Dinitro-2-biphenylamine 98%
10-(2′,4′-Dinitrophenylazo)-9-phenanthrol
3,5-Dinitrosalicylic acid used in colorimetric determination of
reducing sugars
1,1′-Dioctadecyl-4,4′-bipyridinium dibromide 97%
3,3′-Dioctadecyloxacarbocyanine perchlorate
4-(2-[6-(Dioctylamino)-2-naphthalenyl]ethenyl)-1-(3-
sulfopropyl)pyridinium inner salt ≥95% (HPLC), solid
1,3-Diphenylacetone p-tosylhydrazone 98%
2-Diphenylacetyl-1,3-indandione-1-hydrazone 98%
1,2-Diphenylindole 94%
N-(Diphenylmethylene)glycine tert-butyl ester 98%
2,6-Diphenyl-4H-thiopyran-4-one 96%
Direct Blue 71 Dye content 50%
Direct Blue 15 suitable for Histopaque ® system, suitable for
viability studies of collagenase-treated rat liver cells
Direct Red 23 Dye content 30%
Direct Red 80 Dye content 25%
Direct Red 81 Dye content 50%
Direct yellow 27
Disperse Black 9 Dye content 97%
Disperse Blue 1 Dye content 30%
Disperse Blue 3 Dye content 20%
Disperse Blue 14 Dye content 97%
Disperse Orange 1 Dye content ~15%
Disperse Orange 13 Dye content 90%
Disperse Orange 13 Dye content 15%
Disperse Yellow 3 Dye content 30%
Dithizone Practical Grade
Dithizone ACS reagent, ≥85.0%
E
Eosin B certified by the Biological Stain Commission, Dye content
90%
Eosin B for microscopy (Fl., Hist., adsorption and fluorescent
indicator
Eosin B Dye content 95%
Eosin Y Dye content ~99%
Eosin Y
Eosin Y disodium salt Dye content ≥85%
Eosin Y disodium salt certified by the Biological Stain Commission
Eosin Y solution 5 wt. % in H2O
Eriochrome ® Black T ACS reagent (indicator grade)
Erioglaucine disodium salt
Erythrosin B Dye content ≥95%
Erythrosin B certified by the Biological Stain Commission, Dye
content 90%
Erythrosin extra bluish for microscopy (Bact., Hist.), adsorption
and fluorescent indicator
Erythrosin extra bluish certified by the Biological Stain
Commission
Erythrosin yellowish blend
Ethidium bromide ~95% (HPLC)
Ethidium bromide monoazide ≥95% (HPLC), solid
Ethidium homodimer 1 solution ≥95%, 2 mM in DMSO
5-[3-Ethoxy-4-(3-ethyl-5-methyl-2(3H)-benzothiazolylidene)-2-
butenylidene]-3-ethyl-2-[(3-ethyl-4,5-diphenyl-2(3H)-
thiazolylidene)methyl]-4,5-dihydro-4-oxothiazolium iodide Dye
content 95%
3-Ethoxy-4-methoxybenzaldehyde 99%
2-[3-(3-Ethyl-2(3H)-benzothiazolylidene)-2-methyl-1-propenyl]-3-
[3-(sulfooxy)butyl]benzothiazolium hydroxide inner salt Dye
content 90%
5-Ethyl-5,6-dihydro-3,8-dinitro-6-phenyl-6-phenanthridinol 97%
Ethyl 3,5-dimethyl-2-pyrrolecarboxylate 98%
Ethyl eosin certified by the Biological Stain Commission
Ethyl 4′-hydroxy-4-biphenylcarboxylate 98%
3-Ethyl-2-methylbenzothiazolium iodide
Ethyl Orange sodium salt indicator grade, Dye content 90%
2-(Ethylthio)benzothiazole 97%
2-Ethyl-2-thiopseudourea hydrobromide 98%
N-Ethyl-o-toluidine 97%
Ethyl Violet cationic triarylmethane dye
Ethyl viologen diperchlorate 98%
Evans Blue Dye content ≥75%
F
Fast Black K Salt hemi(zinc chloride) salt practical grade
Fast Blue BB Salt hemi(zinc chloride) salt Dye content ≥80%
Fast Blue BB Salt hemi(zinc chloride) salt for microscopy (Hist.)
Fast Blue RR
Fast Blue RR Salt crystalline
Fast Blue B Salt Dye content ~95%
Fast Corinth V zinc chloride double salt Dye content 90%
Fast Dark Blue R Salt
Fast Garnet GBC sulfate salt diazonium dye
Fast Garnet GBC base 97%
Fast Green FCF Dye content ≥85%
Fast Green FCF certified by the Biological Stain Commission
Fast Red B tetrafluoroborate salt Dye content 95%
Fast Red ITR
Fast Red RC Salt
Fast Red Violet LB base
Fast Red Violet LB Salt Dye content ≥90%
Fat Brown B
Ferroin indicator solution 0.1 wt. % in H2O
FIM-1
FIM-1 diacetate
Fluorescein sodium salt used as fluorescent tracer
Fluorescein-5-EX N-hydroxysuccinimide ester
Fluoresceinamine, isomer I
Fluorescein diacetate used as cell viability stain
Fluorescein (free acid) Dye content 95%
Fluorescein 5(6)-isothiocyanate ≥90% (HPLC)
Fluorescein isothiocyanate isomer I suitable for protein labeling,
≥90% (HPLC), powder
Fluorescein isothiocyanate isomer I ≥97.5% (HPLC)
Fluorescein isothiocyanate isomer I-Celite ® suitable for
fluorescent labeling techniques
Fluorescein Sodium salt - CAPS solution for fluorescence, ≥95.0%
(HPLC)
Fluorescent Brightener 28 used as a stain and brightening agent
Fluorinert ™ FC-40
4-Formylbenzene-1,3-disulfonic acid disodium salt hydrate 97%
Fura 2-AM ≥95% (HPLC)
Fura-2 LeakRes (AM) ≥85%
Fusidic acid
Plasmocorinth B Dye content 60%
G
2-(β-D-Galactosidoxy)naphthol AS-LC
Gallocyanine Dye content 90%
Gentian violet for microscopy (Bact., Hist.)
Gentian Violet meets USP testing specifications
Giemsa stain technical grade, used as a blood stain
Giemsa stain certified by the Biological Stain Commission
Giemsa Stain, Modified Solution (for the staining (of
cellular blood components and blood parasites))
Glutaraldehyde bis(2,4-dinitrophenylhydrazone) analytical
standard, for environmental analysis
Guinea Green B Dye content 50%
H
H + LC:L[49]Cematein for microscopy (Hist.)
Hematoxylin
Hematoxylin certified by the Biological Stain Commission
1-Heptyl-4-(4-pyridyl)pyridinium bromide 95%
6-Hexadecanoyl-2-(((2-
(trimethylammonium)ethyl)methyl) amino)naphthalene
chloride solid
4′-Hydroxy-4-biphenylcarboxylic acid 99%
1-(2-Hydroxyethyl)-1,2,3,4-tetrahydro-2,2,4,7-
tetramethylquinoline 97%
8-Hydroxy]ulolidine 97%
Hydroxy naphthol blue disodium salt ACS reagent
2-Hydroxy-1,4-naphthoquinone 97%
2-(4-Hydroxyphenyl)-5-pyrimidinol 90%
1-(4-Hydroxyphenyl)-2,4,6-triphenylpyridinium hydroxide
inner salt hydrate 97%
I
Immersion oil viscosity 150 cSt (lit.)
Immersion oil viscosity 1,250 cSt (lit.)
Indigo synthetic, Dye content 95%
Indigo carmine for microscopy (Bact., Hist.), indicator (pH
11.5-14.0)
Indigo carmine certified by the Biological Stain
Commission, Dye content 85%
Indophenol
Indophenol Blue Dye content 60%
Indoxyl β-D-galactopyranoside
Indulin B practical grade
1-Iodo-3,5-dinitrobenzene 98%
Iodonitrotetrazolium chloride Used in colorimetric assays.
5-Iodosalicylaldehyde 97%
IR-797 chloride Dye content 70%
Isopentyl nitrite 96%
4-(4-isothiocyanatophenylazo)-N,N-dimethylaniline 97%
J
Janus Green B certified by the Biological Stain
Commission, Dye content 65%
JC-1 solid
Jenner's stain suitable for blood stain
Jenner's stain certified by the Biological Stain Commission
K
Keratin azure
L
Lacmoid
Leishman's stain used as histology stain
Leit-Silver for electron microscopy
Leucoberbelin Blue I Dye content 65%
Leucocrystal Violet
Leucomalachite Green
Light Green SF Yellowish crystalline
Light Green SF Yellowish certified by the Biological Stain
Commission
Lissamine ™ Green B Dye content 60%
Lithium carbonate puriss. p.a., ACS reagent, reagent (for
microscopy), ≥99.0% (T)
LR white acrylic resins Medium
Lucifer Yellow CH dilithium salt fluorescent stain
Lucifer Yellow VS dilithium salt ~85%
Lugol solution for microscopy (Bact., Bot.)
Lumichrome
M
Malachite Green oxalate salt Technical grade
Malachite Green oxalate salt certified by the Biological
Stain Commission
Malachite Green Carbinol hydrochloride Dye content 85%
Malonaldehyde bis(phenylimine) monohydrochloride 97%
Martius Yellow Dye content 85%
Martius Yellow sodium salt monohydrate 98%
May-Grünwald Stain
Melanin from Sepia officinalis
Metanil Yellow for microscopy (Hist.), indicator (pH 1.2-
2 3)
Metanil Yellow Dye content 70%
4-(4-Methoxybenzylamino)-7-nitrobenzofurazan
4-Methoxybiphenyl 97%
3-Methoxydiphenylamine 98%
1-Methoxy-S-methylphenazinium methyl sulfate ≥95%
2-Methoxy-N4-phenyl-1,4-phenylenediamine 95%
6-Methoxy-1,2,3,4-tetrahydro-9H-pyrido[3,4-b]indole
97%
6-Methoxy-1,2,3,4-tetrahydro-9H-pyrido[3,4-b]indole
hydrochloride 97%
6-Methoxy-1,2,3,4-tetrahydro-9H-pyrido[3,4-b]indole-1-
carboxylic acid 97%
2-Methyl-2-adamantanol 97%
1-(Methylamino)anthraquinone 98%
Methyl 3-amino-5,6-dichloro-2-pyrazinecarboxylate 97%
Methyl Blue
Methyl Blue certified by the Biological stain Commission
4,4′-Methylenebis(N,N-dimethylaniline) 98%
Methylene blue certified by the Biological Stain
Commission
Methylene Blue hydrate suitable for nucleic acid staining,
BioReagent
Methylene Blue solution for microscopy, concentrate
according to Ehrlich, concentrated, aqueous solution
Methylene Blue solution 1.4% (w/v) in 95% ethanol
Methylene Blue solution for microbiology
4,5-Methylenedioxy-1,2-phenylenediamine
dihydrochloride Fluorogenic reagent
Methylene Violet (Bernthsen) certified by the Biological
Stain Commission, Dye content ≥65%
Methylene Violet 3RAX Dye content 90%
Methyl Green zinc chloride salt, ~85%
Methyl Green zinc chloride salt, for microscopy (Bact.,
Bot., Hist.)
Methyl Green zinc chloride salt, certified by the Biological
Stain Commission, Dye content 85%
Methyl Green zinc chloride salt, <0.5% crystal violet, Dye
content 80%
4-Methyl-3-nitrobenzyl chloride 97%
Methyl Orange ACS reagent, Dye content 85%
4-Methylphthalonitrile 99%
Methyl Purple in H2O
Methyl Red ACS reagent, crystalline
Methyl Red hydrochloride ACS reagent
Methyl Red sodium salt Crystalline
Methyl Red sodium salt ACS reagent, Dye content 95%
Methyl violet 2B certified by the Biological Stain
Commission
Methyl Violet B base Dye content 85%
Mordant Blue 9 Dye content 50%
Mordant Orange 1 Dye content 70%
Morin hydrate for microscopy (Fl.), for the determination
of Al, Be, Zn, Ga, In, Sc, 1-2 mol/mol water
MIT Formazan powder
Murexide ACS reagent
N
2,3-Naphthalenedicarbonitrile 97%
2,3-Naphthalenedicarboximide 97%
α-Naphtholbenzein indicator (pH 8.2-10.0)
α-Naphtholbenzein indicator grade
Naphthol Blue Black Dye content ~50%
Naphthol Blue Black Dye content 80%
Naphthol Green B Technical grade
Naphthol Green B for microscopy (Hist.), for
complexometry
Naphthol AS-GR phosphate disodium salt
Naphthol AS-MX phosphate disodium salt phosphatase
substrate
Naphthol AS phosphate
Naphthol AS phosphate disodium salt
α-Naphtholphthalein practical grade
Naphthol Yellow S for microscopy (Hist.), for the
precipitation (of amino acids and peptides)
1-Naphthyl red hydrochloride Dye content 85%
Neutral Red powder, BioReagent, suitable for cell culture
Neutral Red Dye content ≥90%
Neutral Red certified by the Biological Stain Commission
New Coccine Dye content 75%
Nigrosin certified by the Biological Stain Commission
Nigrosin water soluble For use as a biological stain
Nile Blue A Dye content ≥75%
Nile Blue A certified by the Biological Stain Commission
Nile Red Technical grade
Nitrazine Yellow indicator grade, Dye content 85%
4-Nitroazobenzene technical grade, 90%
3-Nitrobenzaldoxime 99%
4-Nitrobenzenediazonium tetrafluoroborate 97%
4-Nitroguaiacol 97%
4-Nitroguaiacol potassium salt hydrate 98%
4-(4--Nitrophenylazo)resorcinol Dye content 90%
2-(3-Nitrophenylsulfonyl)ethanol 97%
4-Nitrophthalamide 99%
3-Nitrophthalimide 97%
4-Nitrophthalimide 98%
3-Nitrophthalonitrile 99%
4-Nitrophthalonitrile 99%
Nitrotetrazolium Blue chloride ~98% (TLC)
Nitrotetrazolium Blue chloride powder, electrophoresis
grade
Nitrotetrazolium Blue chloride monohydrate
3-Noradamantanamine hydrochloride 95%
3-Noradamantanecarboxylic acic 97%
Nuclear Fast Red
Nuclear Fast Red for microscopy (Bot., Hist.)
Nuclear Fast Red
O
Octyl acetate ≥99%
Oil Blue N Dye content 96%
Oil Red O certified by the Biological Stain Commission
Oil Red O solution 0.5% in isopropanol
Oil Red O solution 0.5% in propylene glycol
Oil Red EGN
Orange G for NA electrophoresis
Orange G certified by the Biological Stain Commission
Orange G certified by the Biological Stain Commission,
Dye content 80%
Orange II sodium salt (Certified by the Biological Stain
Commission), Dye content ≥85%
Orange OT Dye content 75%
Orcein synthetic
Orcein synthetic, certified by the Biological Stain
Commission
Orcinol monohydrate colorimetric detection reagent
Osmium tetroxide Sealed ampule.
Oxacillin sodium salt monohydrate
P
Para Red Dye content 95%
Pararosaniline hydrochloride
Pararosaniline acetate Dye content 90%
Pararosaniline Base Dye content 95%
Pararosaniline Base crystalline
Patent Blue VF Dye content 50%
PDAM for HPLC derivatization
4-Pentylbicyclo[2.2.2]octane-1-carboxylic acid 99%
Perylene sublimed grade, ≥99.5%
Perylene ≥99%
Phenazine ethosulfate ≥95%
Phenolphthalein puriss., meets analytical specification of
Ph Eur., BP, 98-101% (calc. to the dried substance)
Phenolphthalein ACS reagent
Phenolphthalein solution 0.5 wt. % in ethanol:water (1:1)
Phenolphthalein bisphosphate tetrasodium salt ~95%
Phenol Red powder, BioReagent, suitable for cell culture
Phenol Red ACS reagent
Phenol Red sodium salt powder, BioReagent, suitable for
cell culture, suitable for insect cell culture
Phenol Red sodium salt
Phenol Red sodium salt ACS reagent. Dye content 90%
Phenol red solution 0.5%, liquid, sterile-filtered,
BioReagent, suitable for cell culture
Phenosafranin Dye content 80%
3-Phenoxyphthalonitrile 98%
4-Phenoxyphthalonitrile 98%
N-[5-(Phenylamino)-2,4-pentadienylidene]aniline
monohydrochloride 98%
4-(Phenylazo)diphenylamine 97%
4-Phenylazophenol 98%
1-Phenyl-2,3-naphthalenedicarboxylic anhydride 98%
5-Phenyl-2-[2-[[5-phenyl-3-(3-sulfoproprl)-2(3H)-
benzoxazolylidene]methyl]-1-butenyl]-3-(3-
sulfopropyl)benzoxazolium hydroxide inner salt, sodium
salt Dye content 90%
2-(Phenylsulfonyl)ethanol 97%
Phloroglucinol Used to detect the presence of wood fiber.
Phloxine B antibacterial fluorescent dye
Phloxine B Dye content ≥80%, certified by the Biological
Stain Commission
Pinacyanol bromide Dye content 95%
Pinacyanol chloride
Poly(1-methoxy-4-(O-disperse Red 1))-2,5-
phenylenevinylene
Ponceau S BioReagent, suitable for electrophoresis
Ponceau S for microscopy (Hist.)
Ponceau S Dye content 75%
Ponceau S solution BioReagent, suitable for
electrophoresis, 0.1% (w/v) in 5%, acetic acid
Ponceau BS Dye content ~60%
Ponceau SS Dye content 80%
Ponceau Xylidine Dye content ≥60%
Potassium indigotetrasulfonate Dye content 85%
Potassium indigotrisulfonate ozone-scavenging reagent
Procion ® Red MX-5B Dye content 40%
Proflavine hemisulfate salt hydrate powder
Propidium iodide ≥94.0% (HPLC)
Propidium iodide solution solution (1.0 mg/ml in water)
Propionaldehyde-2,4-dinitrophenylhydrazone analytical
standard, for environmental analysis
Prussian blue soluble for microscopy
Purpurin Dye content 90%
1-Pyrenebutyric acid N-hydroxysuccinimide ester 95%
3,4-Pyridinedicarbonitrile 96%
3,4-Pyridinedicarboxamide 98%
l-(2-Pyridylazo)-2-naphthol indicator grade
4-(2-Pyridylazo)resorcinol 96%
4-(2-Pyridylazo)resorcinol monosodium salt hydrate
3-(2-Pyridyl)-5,6-di(2-furyl)-1,2,4-triazine-5′,5″-disulfonic
acid disodium salt
3-(2-Pyridyl)-5,6-diphenyl-1,2,4-triazine-4′,4″-disulfonic
acid sodium salt BioXtra
Pyrocatechol Violet suitable for indicator
Pyrogallol Red
Pyronin Y for NA electrophoresis
Pyronin B Dye content ≥30%
Pyronin B certified by the Biological Stain Commission,
Dye content 40%
Pyronin Y for microscopy (Bot., Fl., Hist.)
Pyronin Y certified by the Biological Stain Commission,
Dye content 50%
Q
Qdot 525
Qdot 565
Qdot 605
Qdot 655
Qdot 705
Qdot 800
Quinaldine Red Dye content 95%
Quinoline Yellow for microscopy (Hist.), mixture of mono-
and disulfonic acid sodium salt
Quinoline Yellow Dye content 95%
Quinoline Yellow Mixture of the mono- and disulfonic
acids of Quinoline Yellow
R
Reactive Black 5 Dye content ≥50%
Reactive Blue 4 Dye content 35%
Reactive Green 19 practical grade
Reactive Orange 16 Dye content ≥70%
Reactive Red 120
Reichardt's dye Dye content 90%
Remazol Brilliant Blue R anthraquinone dye
Renin Substrate 1
Resazurin sodium salt certified by the Biological Stain
Commission
Resorcinol BioXtra, ≥99%
Resorufin Dye content 95%
Rhodamine 6G Dye content 99%
Rhodamine 6G Dye content ~95%
Rhodamine 6G perchlorate Dye content 99%
Rhodamine B ≥95% (HPLC)
Rhodamine 110 chloride Dye content ≥88%
Rhodamine 123 mitochondrial specific fluorescent dye
Rhodamine B base Dye content 97%
Rhodamine B isothiocyanate-Dextran average mol wt
~10,000
Rhodamine B isothiocyanate mixed isomers
Rhodanile Blue A complex of Nile Blue and Rhodamine B.,
Dye content 75%
RIM-1 ≥90% (HPLC)
Rose bengal Dye content 95%
Rose Bengal sodium salt Dye content ≥85%
Rose Bengal diacetate
Sosa Bengal lactone 95%
p-Rosolic acid Dye content 85%
Rubrene powder
Ruthenium Red Technical grade
Ruthenium Red for microscopy, ≥85% (calc. on dry
substance, AT)
S
Saffron crude source of crocetin and crocein
Safranin O Dye content ≥85%
Safranin O certified by the Biological Stain Commission
Safranin O for microscopy (Bact., Bot., Hist.), indicator (pH
0.3-1.0)
Schiff's fuchsin-sulfite reagent suitable for detection of
glycoproteins
Scopoletin ≥99%
Silver diethyldithiocarbamate
Silver Enhancer Kit
Silver enhancer solution A
Silver enhancer solution B
Silver nitrate BioXtra, >99% (titration)
Silver nitrate ReagentPlus ®, ≥99.0% (titration)
Silver proteinate ~8% Ag basis
Solvent Blue 38 practical grade
Solvent Blue 59 Dye content 98%
Solvent Green 3 Dye content 95%
Stains-All ~95%
trans-4-Stilbenemethanol
Sudan I Dye content ≥95%
Sudan II Dye content 90%
Sudan III Technical grade
Sudan III certified by the Biological Stain Commission,
BioXtra
Sudan IV certified by the Biological Stain Commission,
BioXtra
Sudan IV Dye content ≥80%
Sudan Black B certified by the Biological Stain Commission
Sudan Blue II Dye content 98%
Sudan Orange G Dye content 85%
Sudan Red 7B for microscopy (Bot., Hist.)
Sudan Red 7B Dye content 95%
Sulfanilic acid azochromotrop ≥80%
2-Sulfobenzoic acid cyclic anhydride technical grade, 90%
Sulfochlorophenol S sodium calcium salt
Sulforhodamine 101
Sulforhodamine B sodium salt Technical grade
Sunset Yellow FCF Dye content 90%
SYBR ® Green II RNA gel stain 10,000 × in DMSO Green
Alternative
SYBR ® Green I nucleic acid gel stain 10,000 × in
DMSO Green Alternative
SynaptoGreen ™ C4 ≥95% (HPLC), solid
SynaptoRed ™ C2 ≥95% (HPLC), solid
Syringaldazine 99%
T
Tannic acid Source: Chinese natural gall nuts
Tetrabromophenol Blue Dye content 85%
Tetrabromophenol Blue sodium salt Dye content 85%
3′,3″,5′,5″-Tetrabromophenolphthalein ethyl ester
potassium salt indicator grade
3,4,5,6-Tetrabromophenolsulfonephthalein Dye content
95%
Tetrabromo-2-sulfobenzoic acid cyclic anhydride
Tetrabutylammonium bis(3,6-dichloro-1,2-
benzenedithiolato)nickelate 98%
Tetrabutylammonium bis(4-methyl-1,2-
benzenedithiolato)nickelate 98%
Tetrachlorophthalonitrile 98%
Tetrachrome Stain (MacNeal)
2,3,6,7-Tetrahydro-8-hydroxy-1H,5H-benzo[ij]quinolizine-
9-carboxaldehyde 98%
1,2,3,4-Tetrahydro-2,2,4,7-tetramethylquinoline 97%
3′,3″,5′,5″-Tetraiodophenolsulfonephthalein Dye content
90%
1,2,3,3-Tetramethyl-3H-indolium iodide 98%
2,2,4,4-Tetramethyl-3-pentanone imine 95%
N,N,N′,N′-Tetramethyl-p-phenylenediamine
dihydrochloride ≥95%, powder
Tetramethylrhodamine-5-isothiocyanate
Tetramethylrhodamine methyl ester perchlorate ≥95%
Tetranitroblue tetrazolium chloride
Tetrazolium Blue Chloride used in colorimetric
determination of reducing compounds
Tetrazolium Violet
TFLZn potassium salt ≥90% (TLC)
TFLZn-AM ≥90% (TLC)
Thiazole Orange Dye content ~90%
Thiazolyl Blue Tetrazolium Bromide 98%
Thioflavine S practical grade
Thioflavin T used as stain for amyloid
Thionin acetate salt certified by the Biological Stain
Commission, Dye content ≥85%
Thymol Blue ACS reagent
Thymol Blue sodium salt ACS reagent, Dye content 95%
Thymolphthalein ACS reagent, Dye content 95%
Thymoquinone ≥98%
o-Tolidine ≥97%, powder
Toluidine Blue O Technical grade
Toluidine Biue O certified by the Biological Stain
Commission
Toluidine Red Dye content 70%
3,4,5-Trihydroxybenzamide 98%
Tris(4-nitrophenyl)amine technical grade
Trypan Blue Dye content 60%
Trypan Blue powder, BioReagent, suitable for cell culture
Trypan Blue solution 0.4%, liquid, sterile-filtered, suitable
for cell culture
U
Uniblue A sodium salt
V
Valeraldehyde-2,4-dinitrophenylhydrazone environmental
standard, 99%
Vanillin azine 99%
Variamine Blue RT Salt
Victoria Blue R Dye content 80%
Victoria Pure Blue BO Dye content 90%
Violamine R Dye content 50%
W
Wright stain suitable for blood stain
Wright stain certified by the Biological Stain Commission
Wright Stain solution for microscopy
X
p-Xylene-bis(N-pyridinium bromide) ≥95% (TLC)
Xylene Cyanol FF Dye content ≥75%
Xylene Cyanol FF for molecular biology, BioReagent
Xylenol Blue indicator grade, Dye content 90%
Xylenol Orange terrasodium salt ACS reagent
Xylidyl blue I
Z
Zincon sodium salt Dye content ≥75%
Zinquin ≥95% (HPLC), solid
Zinquin ethyl ester ≥95% (HPLC), solid

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The term “comprising” whenever used in this document is intended to indicate the presence of stated features, integers, steps, components, but not to preclude the presence or addition of one or more other features, integers, steps, components or groups thereof.

The disclosure is of course not in any way restricted to the embodiments described and a person with ordinary skill in the art will foresee many possibilities to modifications thereof without departing from the basic idea of the disclosure as defined in the appended claims.

The above described embodiments are obviously combinable.

The following dependent claims set out particular embodiments of the disclosure.

SEQUENCE LISTING
SEQ- ID. NO. 1:
Folic acid-DRDDQAAWFSQY
SEQ- ID. NO 2:
KDEPQRRSARLSAKPAPPKPEPKPKKAPAKK-DRDDQAAWFSQY
SEQ- ID. NO 3:
YQSFWAAQDDRD-KDEPQRRSARLSAKPAPPKPEPKPKKAPAKK

### REFERENCES

[1] Wang, A. Z., Langer, R., and Farokhzad, O. C., Nanoparticle Delivery of Cancer Drugs, Annual Review of Medicine. 2012, 63, 185-198.
[2] Hong, M.-S., Lim, S.-J., Lee, M.-K., Kim, Y. B., and Kim, C.-K., Prolonged Blood Circulation of Methotrexate by Modulation of Liposomal Composition, Drug Delivery. 2001, 8, 231-237.
[3] Laouini, A., Jaafar-Maalej, C., Limayem-Blouza, I., Sfar, S., Charcosset, C., and Fessi, H., Preparation, Characterization and Applications of Liposomes: State of the Art, Journal of Colloid Science and Biotechnology. 2012, 1, 147-168.
[4] Bangham, A. D., Standish, M. M., and Watkins, J. C., Diffusion of univalent ions across the lamellae of swollen phospholipids, Journal of Molecular Biology. 1965, 13, 238-IN27.
[5] Batzri, S. and Korn, E. D., Single bilayer liposomes prepared without sonication, Biochimica et Biophysica Acta (BBA)-Biomembranes. 1973, 298, 1015-1019.
[6] Naeff, R., Feasibility of topical liposome drugs produced on an industrial scale, Advanced Drug Delivery Reviews. 1996, 18, 343-347.
[7] Sur, S., Fries, A. C., Kinzler, K. W., Zhou, S., and Vogelstein, B., Remote loading of preencapsulated drugs into stealth liposomes, Proceedings of the National Academy of Sciences of the United States of America. 2014, 111, 2283-2288.
[8] Gubernator, J., Active methods of drug loading into liposomes: Recent strategies for stable drug entrapment and increased in vivo activity. Vol. 8. 2011. 565-80.
[9] Duong, A. D., Collier, M. A., Bachelder, E. M., Wyslouzil, B. E., and Ainslie, K. M., One Step Encapsulation of Small Molecule Drugs in Liposomes via Electrospray-Remote Loading, Mol Pharm. 2016, 13, 92-9.
[10] Jaafar-Maalej, C., Diab, R., Andrieu, V., Elaissari, A., and Fessi, H., Ethanol injection method for hydrophilic and lipophilic drug-loaded liposome preparation, J Liposome Res. 2010, 20, 228-43.
[11] Pons, M., Foradada, M., and Estelrich, J., Liposomes obtained by the ethanol injection method, International Journal of Pharmaceutics. 1993, 95, 51-56.

1. A method for encapsulating an active ingredient in a liposome comprising the following steps:
preparing an ethanolic phase by mixing hydrophobic molecules of phospholipids and a steroid with ethanol,
preparing an aqueous phase with an active ingredient and a targeting agent in a buffer solution;
obtaining liposomes by injecting the ethanolic phase in the aqueous phase, at a temperature from about 50° C. to about 80° C., wherein the ethanolic/aqueous phase volume ratio is between 1:1 and 3:2;
removing ethanol;
removing the remaining free active ingredient by tangencial flow filtration;
wherein the targeting agent is a peptide that is conjugated with a liposomal component or incorporated in a lipidic membrane.

2. The method according to claim 1, wherein the ethanol removal is by evaporation or tangential flow filtration.

3. The method according to claim 1, wherein the steroid is cholesterol.

4. The method according to claim 3, wherein the cholesterol is cholesteryl hemisuccinate.

5. (canceled)

6. The method according to claim 1, wherein the ethanolic phase is injected at a rate of about 2-4 ml/minute.

7. The method according to claim 1, wherein the ethanol concentration, relative to an initial volume of the aqueous phase, is between 40% and 60%.

8. The method according to claim 1, wherein the temperature is 60° C. or 70° C.

9. The method according to claim 1, wherein the active ingredient is a polycharged molecule containing at least one negative charge at a pH of about 4 to about 7.

10. The method according to claim 1, wherein the active ingredient is an anticancer drug, antirheumatic drug, anti-neurodegenerative diseases drug, antioxidant drug, anti-inflammatory; drug, antipyretic drug, antibiotic drug, antiviral drug, analgesic drug or combinations thereof.

11. The method according to claim 1, wherein the injecting step is performed under agitation.

12. The method according to claim 1, wherein the targeting agent comprises a peptide having an amino acid sequence at least 90% identical to the following sequence: SEQ ID NO: 1, SEQ ID NO: 2, SEQ ID NO: 3, or mixtures thereof.

13. The method according to claim 1, wherein the aqueous phase is phosphate buffered saline (PBS).

14. The method according to claim 1, wherein the ethanolic phase comprises anionic, neutral, or cationic phospholipids.

15. The method according to claim 1, wherein the phospholipids are selected from the group consisting of: phosphatidylcholines, phosphatidylethanolamines, phosphatidylserines, phosphatidylglycerols, and derivates or mixtures thereof.

16. The method according to claim 1, wherein the ethanolic phase further comprises a stealth agent, the targeting agent, or a mixture of thereof.

17. The method according to claim 16, wherein the stealth agent is polyethylene glycol, PEG, or gangliosides.

18. The method according to claim 17, wherein the polyethylene glycol or PEG, is bound to a phospholipid.

19. (canceled)

20. The method according to claim 1, wherein the active ingredient is an imaging or therapeutic agent.

21. (canceled)

22. The method according to claim 20, wherein the imaging agent is a dye.

23. The method according to claim 1, wherein the active ingredient is methotextrate, doxorubicin or a mixture thereof.


SEQUENCE LISTING (3 sequences)

SEQ ID NO: 1 - protein, 12 aa, Artificial Sequence
  Description of Artificial Sequence Synthetic peptide
  Folic Acid with SEQ ID 1
DRDDQAAWFSQY

SEQ ID NO: 2 - protein, 43 aa, Artificial Sequence
  Description of Artificial Sequence Synthetic polypeptide
  Sequence 2
KDEPQRRSARLSAKPAPPKPEPKPKKAPAKKDRDDQAAWFSQY

SEQ ID NO: 3 - protein, 43 aa, Artificial Sequence
  Description of Artificial Sequence Synthetic polypeptide
  Sequence 3
YQSFWAAQDDRDKDEPQRRSARLSAKPAPPKPEPKPKKAPAKK